(19) United States
(12) Patent Application Publication
(10) Pub. No.: US 2014/0134569 A1
(43) Pub. Date: May 15, 2014

(54) Sodium fluoride, organic powders and aqueous slurry toothpaste formula free flow dental treatment systems

(71) Applicant: Robert Richard Matthews, Washington, DC (US)

(72) Inventor: Robert Richard Matthews, Washington, DC (US)

(21) Appl. No.: 13/675,869

(22) Filed: Nov. 13, 2012

(51) Int. Cl. A61C 1/08; A61K 8/21; A61C 17/16; A61C 1/00; A61C 17/02; A61C 17/022

(52) U.S. Cl. CPC A61C 1/087; A61C 17/0202; A61C 17/022; A61C 17/0208; A61C 17/16; A61C 1/0061; A61K 8/21
USPC 433/84; 433/88

(57) ABSTRACT

The FDA approved toothpaste sodium fluoride, organic powders and aqueous slurry toothpaste formula dental treatment systems are designed to safely clean teeth by physico-chemical means contrasted with highly fluid water picks and toothbrush bristles that can't always reach hard to clean areas, between teeth, and inside molars and crowns. Once tartar and plaque set on teeth they are increasingly more difficult to remove with water picks, dental floss, and toothbrush bristles alone that simply can't remove stubborn residues from many hidden and hard to reach areas of your teeth. The FDA approved toothpaste sodium fluoride, organic powders and aqueous slurry toothpaste formula free flow dental treatment systems subject to the approval of the American Dental Association ADA are designed to safely clean teeth gently with clinical specifications of pressurized air of fine toothpaste powders and clinical specifications of aqueous slurry toothpaste formula of highly specialized shape and rotating nozzles.

### DOMESTIC PRIORITY DATA

This is an application a continuation in part of preliminary amendment to specification application Ser. No. 13/854,508. Filing date Jul. 18, 2011.

### BACKGROUND OF THE INVENTION

The FDA approved toothpaste sodium fluoride, organic powders and aqueous slurry toothpaste formula free flow dental treatment systems are designed to safely clean teeth by physico-chemical means contrasted with highly fluid water picks and toothbrush bristles that can't always reach hard to clean areas, between teeth, and inside molars and crowns.

### BRIEF SUMMARY OF THE INVENTION

Once tartar and plaque set on teeth they are increasingly more difficult to remove with water picks, dental floss, and toothbrush bristles alone that simply can't remove stubborn residues from many hidden and hard to reach areas of your teeth. The FDA approved toothpaste sodium fluoride, organic powders and aqueous slurry toothpaste formula free flow dental treatment systems subject to the approval of the American Dental Association ADA are designed to safely clean teeth gently with clinical specifications of pressurized air with fine toothpaste powders and clinical specifications of aqueous slurry toothpaste formula with highly specialized shape and rotating nozzles.

### BRIEF DESCRIPTION OF THE SEVERAL VIEWS OF THE DRAWING FOLLOWS FIGS. 1 TO 7

FIG. 1 Sodium fluoride, and organic powders toothpaste formula free flow system sizes may vary;

FIG. 2 Sodium fluoride, and organic powders toothpaste formula free flow system sizes may vary;

FIG. 3 Sodium fluoride, organic powders and aqueous slurry toothpaste formula free flow system sizes may vary;

FIG. 4 Sodium fluoride, organic powders and aqueous slurry toothpaste formula free flow system sizes may vary;

FIG. 5 Sodium fluoride, organic powders and aqueous slurry toothpaste formula encapsulated nozzle free flow system sizes may vary;

FIG. 6 Sodium fluoride, and organic powders toothpaste formula differential pressurized variable control regulation free flow system sizes may vary; and

FIG. 7 Sodium fluoride, and organic powders toothpaste formula differential pressurized variable control regulation free flow system with 4× expansion of control valve or control valve venturi.

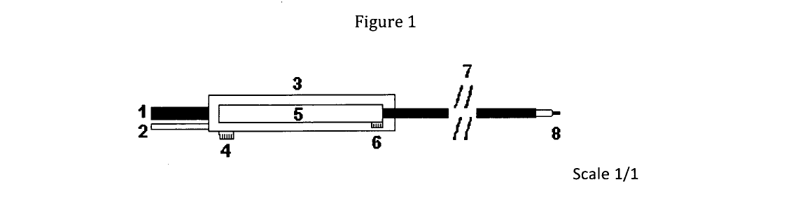

### DETAILED DESCRIPTION OF THE DRAWINGS FIGS. 1 TO 7

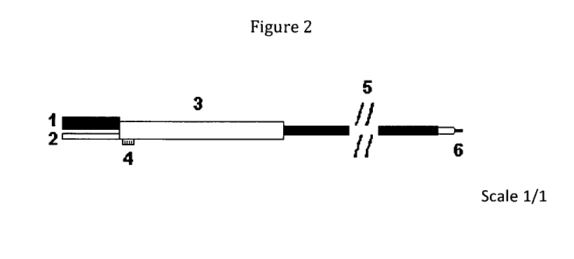

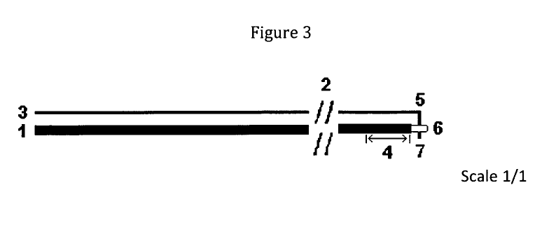

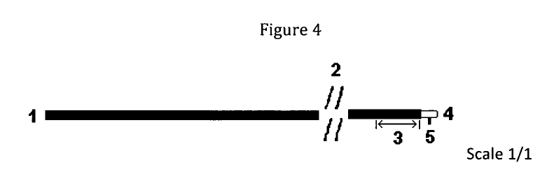

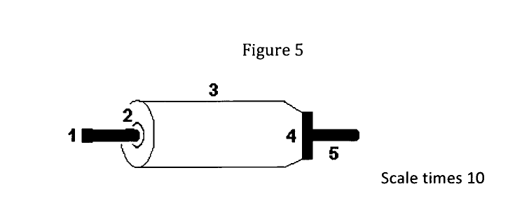

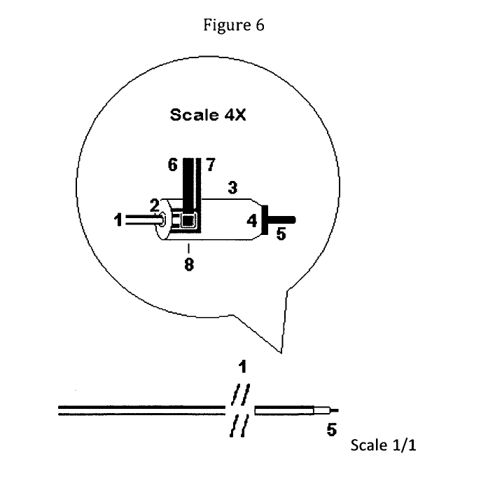

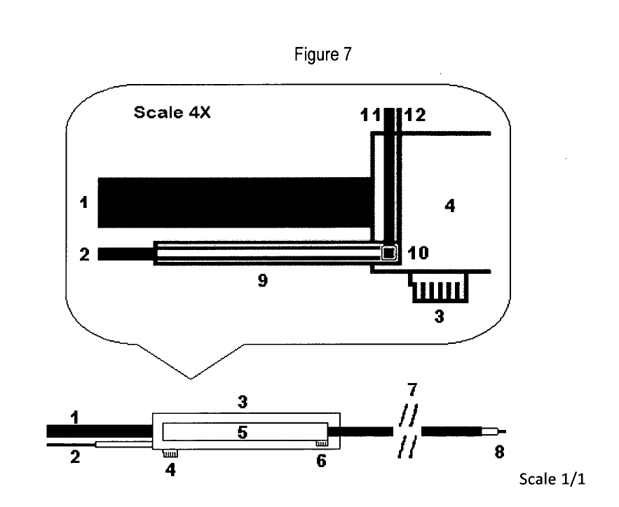

FIG. 1 1—Compressed air hose line-in measured in diameter, 2—Sodium fluoride, and organic powders toothpaste formula hose line-in measured in diameter, 3—Outer pressurized chamber, 4—Variable control regulator, 5—Inner pressurized chamber, 6—Variable control regulator, 7—Compressed air, sodium fluoride, and organic powders toothpaste formula hose line-out measured in diameter, and 8—Encapsulated nozzle sizes may vary, one or two rotating spindles with nozzles, and one or more apertures measured in diameter sizes may vary not shown here, FIG. 2 1—Compressed air hose line-in measured in diameter, 2—Sodium fluoride, and organic powders toothpaste formula hose line-in measured in diameter, 3—Pressurized chamber, 4—Variable control regulator, 5—Compressed air, sodium fluoride, and organic powders toothpaste formula hose line-out measured in diameter, and 6—Encapsulated nozzle sizes may vary, one or two rotating spindles with nozzles, and one or more apertures measured in diameter sizes may vary not shown here, FIG. 3 1—Specification aqueous slurry dental treatment formula pliable and non pliable hose line-in measured in diameter from aqueous slurry dental treatment formula powered pump finite or variable control delimited not shown here; 2—Specification aqueous slurry dental treatment formula pliable and non pliable hose line-in measured in diameter; 3—Specification of pressurized air hose line-in measured in diameter from air compressor pump finite or variable control delimited not shown here; 4—Bi-interval valve measured in diameter not shown here; 5—Specification of pressurized air hose line-in measured in diameter to vertical alignment encapsulated nozzle or standard alignment encapsulated nozzle not shown here; 6—Vertical alignment encapsulated nozzle sizes may vary; and 7—vertical alignment shape nozzle aperture measured in diameter, divergence measured in degrees angulation, or linear, aperture measured in diameter, with three or more sides or continuous measured in diameter sizes may vary not shown here, and one or two rotating spindles with nozzles one or more apertures measured in diameter sizes may vary not shown here, FIG. 4 1—Specification aqueous slurry dental treatment formula pliable and non pliable hose line-in measured in diameter from aqueous slurry dental treatment formula powered pump finite or variable control delimited not shown here; 2—Specification aqueous slurry dental treatment formula pliable and non pliable hose line-in measured in diameter; 3—Bi-interval valve measured in diameter not shown here; 4—Vertical alignment encapsulated nozzle sizes may vary; and 5—vertical alignment shape nozzle aperture measured in diameter, divergence measured in degrees angulation, or linear, aperture measured in diameter, with three or more sides or continuous measured in diameter sizes may vary not shown here, and one or two rotating spindles with nozzles one or more apertures measured in diameter sizes may vary not shown here, FIG. 5 1—Pressurized air, sodium fluoride, organic powders and aqueous slurry toothpaste formula hose line-in measured in diameter, 2—Encapsulated nozzle O-ring regulator pneumatic or fluids connector finite control, 3—Encapsulated nozzle pressurized or fluids chamber, 4—Encapsulated nozzle regulator or fluids breaker 2 aperture measured in diameter, and 5—Nozzle measured in diameter sizes may vary, FIG. 6 1—Compressed air hose line-in measured in diameter; sodium fluoride or organic powders hose line-in measured in diameter, 2—Encapsulated nozzle O-ring regulator pneumatic connector finite control, 3—Encapsulated nozzle pressurized chamber, 4—Encapsulated nozzle regulator 2 aperture measured in diameter, 5—Encapsulated nozzle measured in diameter sizes may vary, 6—control valve sodium fluoride or organic powders hose line-out measured in diameter, 7—Variable compressed air regulator hose line-in measured in diameter control valve, and 8—Control valve, and FIG. 7 Lid with release valve, air filter, silica gel slots, and filtered air gravity feed outlet, 1—Compressed air hose line-in measured in diameter, 2—Sodium fluoride or organic powders hose line-in measured in diameter, 3—Outer pressurized chamber, 4—Variable control regulator, 5—Inner pressurized chamber, 6—Variable control regulator, 7—Compressed air line-out measured in diameter; sodium fluoride or organic powders hose line-out measured in diameter, 8—Encapsulated nozzle, 9—Control valve sodium fluoride or organic powders hose line-in measured in diameter, 10—Control valve, 11—Control valve sodium fluoride or organic powders hose line-out measured in diameter, and 12—Variable compressed air regulator hose line-in measured in diameter control valve.

### DETAILED DESCRIPTION OF THE INVENTION

The sodium fluoride, organic powders and aqueous slurry toothpaste formula free flow dental treatment systems first provide a safe physico-chemical means to clean teeth with sodium fluoride, organic powders and aqueous slurry toothpaste formula.

All parts of the sodium fluoride, organic powders and aqueous slurry toothpaste formula free flow dental treatment systems are built to specification towards the application specificity requirement and conform with the specified requirements to a unique American Society for Testing and Materials ASTM International Standards Worldwide identifiers and sizes may vary.

Description of all parts making and using the same built to specification towards the application specificity requirement sizes may vary for the sodium fluoride, organic powders and aqueous slurry toothpaste formula free flow dental treatment systems 1. Sodium fluoride, organic powders and aqueous slurry toothpaste formula plastic injection molded preformed free flow container sizes may vary for the sodium fluoride, organic powders and aqueous slurry toothpaste formula; 2. Control valve or control valve venturi, specification of negative pressurized air, specification of compressed air control, and specification of pneumatic hose line-in and line-out measured in diameter with or without powered blower specification; 3. Specification pneumatic hose line-in measured in diameter, and specification of compressed air; 4. Outer pressurized chamber, specification of compressed air, and variable control regulator compressed air control; 5. Inner pressurized chamber, specification of compressed air, and variable control regulator compressed air control; 6. Specification pneumatic hose line-out measured in diameter; 7. Encapsulated nozzle, specification of pressurized air or fluids, encapsulated nozzle O-ring regulator pneumatic or fluids connector finite control, encapsulated nozzle regulator 2 specification of compressed air regulated or fluids breaker control, nozzles and rotating spindles attachments measured in diameter, and apertures measured in diameter; 8. Wand with nozzle, and nozzle attachments measured in diameter; 9. Nozzle measured in diameter specifications of pressurized streams and fluids; and 10. Sodium fluoride, organic powders and aqueous slurry dental treatment toothpaste formula.

Description of all parts making and using the same built to specification towards the application specificity requirement sizes may vary for the sodium fluoride, organic powders and aqueous slurry dental treatment toothpaste formula; 1—Specification aqueous slurry dental treatment formula pliable and non pliable hose line-in measured in diameter from aqueous slurry dental treatment formula powered pump finite or variable control delimited not shown here; with or without specification of pressurized air hose line-in measured in diameter from electronic air compressor pump finite or variable control delimited not shown here; and with or without specification of pressurized air hose line-in measured in diameter to vertical alignment encapsulated nozzle or standard alignment encapsulated nozzle not shown here; 2—with or without bi-interval valve measured in diameter not shown here; 3—encapsulated nozzle vertical alignment shape nozzle or vertical alignment shape nozzle without encapsulated nozzle aperture measured in diameter, divergence measured in degrees angulation or linear, aperture measured in diameter, or encapsulated nozzle standard alignment shape nozzle or standard alignment shape nozzle without encapsulated nozzle aperture measured in diameter, divergence measured in degrees angulation or linear, aperture measured in diameter, six to ten sides or continuous measured in diameter not shown here; 4—Plastic injection molded preformed toothbrush wand and toothbrush bristles not shown here; and 5—Plastic injection molded preformed aqueous slurry dental treatment formula dispenser not shown here.

The specification as set forth comprises electrical component systems subject to the approval of the American Dental Association ADA rated alternating AC or direct current DC attached to a portable frame with handles and wheels not shown here; electric motor, air compressor, power supply, rated volts alternating current AC; electric motor, vacuum pump, power supply rated volts alternating current AC; electric motor oscillator, power supply rated volts alternating current AC, or pneumatic oscillator, and pressurized variable control air hose line-in measured in diameter; electric motor, vacuum pump wet or dry, power supply rated volts alternating current AC; and an aqueous slurry dental treatment formula electronic motor pump finite or variable control delimited, power supply rated volts alternating current AC, or direct current DC battery power; electronic air compressor pump finite or variable control delimited, power supply rated volts alternating current AC, or direct current DC battery power not shown here.

The sodium fluoride, organic powders and aqueous slurry toothpaste formula plastic injection molded preformed free flow container utilized specially packaged closed recycled refilled preparation sodium fluoride, organic powders and aqueous slurry toothpaste formula.

The plastic injection molded preformed free flow container variations for sodium fluoride, organic powders and aqueous slurry toothpaste formula for dental treatment may and may not sit in an oscillator not shown here that oscillates to and fro allowing the sodium fluoride, organic powders and aqueous slurry toothpaste formula to evenly distribute while in application.

The sodium fluoride, organic powders and aqueous slurry toothpaste formula plastic injection molded preformed free flow container variations for dental treatment connect directly to the control valve, control valve venturi or to the aqueous slurry toothpaste formula systems FIGS. 1, 2, 3, 4, 6 and 7.

Sodium fluoride, organic powders and aqueous slurry FDA approved toothpaste formula fluids and powders are pumped or drawn into the aqueous slurry and air pressurized systems line-in measured in diameter from an impellar pump or negative pressurized air vacuum pump and transported directly into the encapsulated nozzle fluid system or air pressurized systems control valve or control valve venturi and then projected into the encapsulated nozzle pressure systems where they reach specification diffusion and equal particle distribution.

The sodium fluoride, organic powders and aqueous slurry toothpaste formula exits the encapsulated nozzle into a pressure stream continuous or shape nozzle in addition to one or two rotating spindles with one or more nozzles.

This system comprises air pressurized chambers, variable control regulation, and a dual regulation standard. Differential pressurization is the process obtaining diffusion with equal particle distribution for one, two, or more pressurized streams combined FIGS. 1, 2, 5, 6, and 7.

Differential pressurization can be mathematically determined in cubic feet per minute CFM and pounds per square PSI.

This system comprises an encapsulated nozzle for application of sodium fluoride, organic powders and aqueous slurry toothpaste formula.

1. Description of all parts making and using the same built to specification towards the application specificity requirement sizes may vary for the sodium fluoride, organic powders and aqueous slurry dental treatment toothpaste formula; 1—Specification aqueous slurry dental treatment formula pliable and non pliable hose line-in measured in diameter from aqueous slurry dental treatment formula powered pump finite or variable control delimited not shown here; with or without specification of pressurized air hose line-in measured in diameter from electronic air compressor pump finite or variable control delimited not shown here; and with or without specification of pressurized air hose line-in measured in diameter to vertical alignment encapsulated nozzle or standard alignment encapsulated nozzle not shown here; 2—with or without bi-interval valve measured in diameter not shown here; 3—encapsulated nozzle vertical alignment shape nozzle or vertical alignment shape nozzle without encapsulated nozzle aperture measured in diameter, divergence measured in degrees angulation or linear, aperture measured in diameter, or encapsulated nozzle standard alignment shape nozzle or standard alignment shape nozzle without encapsulated nozzle aperture measured in diameter, divergence measured in degrees angulation or linear, aperture measured in diameter, six to ten sides or continuous measured in diameter not shown here; 4—Plastic injection molded preformed toothbrush wand and toothbrush bristles not shown here; and 5—Plastic injection molded preformed aqueous slurry dental treatment formula dispenser not shown here.

2. The specification as set forth comprises electrical component systems subject to the approval of the American Dental Association ADA rated alternating AC or direct current DC attached to a portable frame with handles and wheels not shown here; electric motor, air compressor, power supply, rated volts alternating current AC; electric motor, vacuum pump, power supply rated volts alternating current AC; electric motor oscillator, power supply rated volts alternating current AC, or pneumatic oscillator, and pressurized variable control air hose line-in measured in diameter; electric motor, vacuum pump wet or dry, power supply rated volts alternating current AC; and an aqueous slurry dental treatment formula electronic motor pump finite or variable control delimited, power supply rated volts alternating current AC, or direct current DC battery power; electronic air compressor pump finite or variable control delimited, power supply rated volts alternating current AC, or direct current DC battery power not shown here.

3. Description of all parts making and using the same built to specification towards the application specificity requirement sizes may vary for the sodium fluoride, organic powders and aqueous slurry toothpaste formula free flow dental treatment systems 1. Sodium fluoride, organic powders and aqueous slurry toothpaste formula plastic injection molded preformed free flow container sizes may vary for the sodium fluoride, organic powders and aqueous slurry toothpaste formula; 2. Control valve or control valve venturi, specification of negative pressurized air, specification of compressed air control, and specification of pneumatic hose line-in and line-out measured in diameter with or without powered blower specification; 3. Specification pneumatic hose line-in measured in diameter, and specification of compressed air; 4. Outer pressurized chamber, specification of compressed air, and variable control regulator compressed air control; 5. Inner pressurized chamber, specification of compressed air, and variable control regulator compressed air control; 6. Specification pneumatic hose line-out measured in diameter; 7. Encapsulated nozzle, specification of pressurized air or fluids, encapsulated nozzle O-ring regulator pneumatic or fluids connector finite control, encapsulated nozzle regulator 2 specification of compressed air regulated or fluids breaker control, nozzles and rotating spindles attachments measured in diameter, and apertures measured in diameter; 8. Wand with nozzle, and nozzle attachments measured in diameter; 9. Nozzle measured in diameter specifications of pressurized streams and fluids; and 10. Sodium fluoride, organic powders and aqueous slurry dental treatment toothpaste formula.
